(19) United States
(12) Patent Application Publication
(10) Pub. No.: US 2016/0067075 A1
(43) Pub. Date: Mar. 10, 2016

(54) FOOT DROP DEVICE

(71) Applicant: Suzanne Malinowski, Windham, NH (US)

(72) Inventors: Suzanne Malinowski, Windham, NH (US); Chad Zrate, Fitchburg, MA (US); Katherine Ann Hamilton, Manchester, NH (US)

(21) Appl. No.: 14/809,630

(22) Filed: Jul. 27, 2015

Related U.S. Application Data
(60) Provisional application No. 62/046,272, filed on Sep. 5, 2014.

(51) Int. Cl. A61F 5/01

(52) U.S. Cl. CPC A61F 5/0113; A61F 2005/0179; A61F 2005/0167

(57) ABSTRACT

This invention describes a multi-point soft flexible ankle foot brace support orthosis apparatus and method for compensating for weakness of the ankle and ankle/foot dorsiflexion resulting in steppage gait of foot drop comprising an ankle cuff that is securely connected to footwear by way of clips and elastic cords, allowing for natural walking motion. The connection of the ankle cuff to the shoe is secure, yet adjustable to accommodate each user's individual need and is easily fastened and unfastened for daily use.

### RELATED APPLICATION

This application claims the benefit of U.S. Provisional Application No. 62/046,272 filed Sep. 5, 2014, incorporated herein by reference in its entirety for all purposes.

### FIELD OF THE INVENTION

The invention relates to the field of foot and ankle support. Particularly, an ankle-foot orthosis apparatus and a method for providing long-term walking assistance compensating for weakness of ankle and toe dorsiflexion resulting in steppage gait of foot drop, or difficulty in lifting the front of the foot off the ground.

### BACKGROUND OF THE INVENTION

Foot drop, also known as drop foot, is a condition commonly characterized by difficulty or inability to lift the front portion of one or both feet during walking or running due to nerve damage causing weakened or paralysis of the muscles in the anterior portion of the lower leg, affecting the function of the foot. Foot drop is compensated for by lifting the foot higher than normally necessary to avoid tripping over the toes. This gait and the compensating movement is known as a steppage gait. Those with foot-drop typically otherwise have sufficient muscular control to move the foot relative to the ankle in plantarflexion (downward motion), but lack muscular control to subsequently raise the foot back up for the next step (dorsiflexion). Also common in persons having foot-drop is a reduced capacity to rotate the outer part of the foot in an upward manner (eversion). Typically, dorsiflexion and eversion are weaker than plantarflexion and inversion (inward turning), requiring support to overcome the resultant gate. Without wearing any type of brace, mobility is greatly decreased, tripping/falling hazards are present and, over time, compensatory walking patterns can cause back, hip, knee, ankle, and foot issues and injuries.

Commonly, rigid, semi/rigid, or hinged braces are supported in the industry by medical/industry professionals as they are covered by insurance. Soft flexible braces are not typically covered by insurance and must be paid for by the user.

Available rigid braces are characterized by limited flexibility, restricting normal and desirable foot and ankle motion resulting in an awkward gait. Although more recent braces may allow for a slightly higher degree of flexibility using light-weight materials, the nature of the rigid brace still presents the same overall concerns. Rigid braces for foot drop are designed to keep the foot and leg at a fixed 90 degree angle at all times, contributing to muscle atrophy from limited movement and use. Without the full range of motion during walking, the fixed ankle position may also lead to compensatory patterns at the knee, hip and within the foot. Additionally, a rigid brace does not allow the leg muscles and ankle to get the important flexibility/exercise needed and loss of muscle, ankle strength, and flexibility is a result. While some rigid braces are advertised as fitting in a normal shoe, they may not, requiring one size larger, extra wide and extra depth shoes. Some users only have one foot affected requiring two different size shoes. Shoe selection to accommodate rigid braces is extremely limited. Sneakers and a limited variety of available shoes with laces or a Velcro® strap (which are characteristically not designed for fashion) fit the brace and hold it in place securely. Velcro® is a registered trademark of Velcro S. A. Corporation Switzerland Lenzerheide, Grisons Switzerland. Wearing dress shoes, heels, sandals, or any shoe without laces, Velcro® strap, or a flat heel is not an option. A user cannot wear pants with a tapered leg as they don't fit over the brace. A fabric sleeve may be needed over the leg to overcome chafing from the brace strap that wraps around the calf. Furthermore, shoes typically need to be modified in some way requiring leather to be added in certain locations to keep shoe material from ripping. The shoe is stretched, and eventually rips inside due to the brace strut and continuous movement of the footplate. This is typical with most braces that sit inside the shoe. Another adaptation for some braces is the need to use elastic shoe laces to keep the laces from breaking due to the continuous movement/pull of the strut. Wearers find that rigid braces tend to break from various repetitive activities requiring bending at the knee and ankle, such as exercise, stair use, running, hiking, walking on uneven terrain, gardening, or riding a bicycle. The rigid braces are often uncomfortable to walk or be active in and many users refuse to wear them. Either way, the result is an inability to participate in many of life's activities.

While available soft braces may be an improvement over rigid braces for many reasons, they are usually not suitable for the wearer that wants to return to their active lifestyle prior to the foot drop condition, such as working full time (fast paced use for 9-10 hours plus per day) or participating in certain activities requiring full confidence that tripping is not a concern. Note that an average of 10,000 steps are taken per day. Since soft braces are not currently supported by insurance or industry professionals, many devices are sold to the wearer with no professional advice or support of the product.

Contrary to advertising, some of these soft braces may not feel trustworthy to provide sufficient support. While simple soft brace designs may exist that provide short term relief from the rigid braces, confidence of use may lack if it is felt that tripping is possible. Among their problems is that most of them address only the lift of the toes and do not address any type of side foot tilt issues that many people have as part of their condition. Specific components of existing soft braces that can cause concern include the number and location of connection points from the ankle brace to the shoe. Most are not positioned properly or securely to provide the appropriate foot lift to prevent tripping for long term use. In some cases, the brace is pulled down toward the foot and does not hold the 90 degree angle very long. Parts pull out of the shoe or loosen the laces they are connected to, and hook and loop fasteners (Velcro®) do not hold securely after a short time. On some braces, the tension is not adjustable for use in various shoes. As with rigid braces, there is a limited shoe selection as most work with only shoes that have shoe laces or eyelets. Wearing dress shoes, heels, sandals, boots, or slippers—any shoe without laces or Velcro® strap is not an option with any type of brace available on the market. In an attempt to make the soft braces easily sellable, they fall short by using hooks or existing parts of the shoes for connection, without installation required. Although this does seem to make the product user friendly, it does not provide the reliability needed by the user.

What is needed, therefore, is a soft foot-assist device that is lightweight, relatively inconspicuous, easy to use, comfortable, inexpensive, provides dorsiflexion and eversion support for the foot that is durable and suitable for long-term wearing, athletic activities, and with various shoe types. Furthermore, this type of brace would allow for full range of motion of the foot and ankle, increased flexibility, mobility, and reliability.

### SUMMARY OF THE INVENTION

An embodiment provides a flexible ankle foot orthosis device for treating foot drop steppage gait and providing ankle support comprising an ankle component; a foot component; a plurality of ankle attachment components disposed on the ankle component; a plurality of foot attachment components disposed on the foot component; and elastic tension components between the ankle attachment components and the foot attachment components; wherein the elastic tension components at the ankle attachment components are adjustable before and during wearing. In embodiments three ankle attachment components are disposed on the ankle component and three foot attachment components are disposed on the foot component. In other embodiments, each of the ankle attachment components comprises a clip configured to receive at least one elastic tension component. In subsequent embodiments, each clip has a serpentine path retaining at least one elastic tension component. For additional embodiments, the serpentine path comprises at least one aperture, at least one prong, and at least one rear clip, whereby at least one elastic component can be drawn to adjust and maintain tension between the ankle component and the foot component. In another embodiment, the serpentine path comprises two apertures, two rear clips, and two prongs. For a following embodiment, adjustment is accomplished by a single pulling motion with one hand on at least one elastic tension component. In subsequent embodiments the ankle attachment component comprises a clip top component and a clip bottom component. In additional embodiments, the clip top component is affixed to the ankle component, the clip bottom component removably attaches to the clip top component and comprises two clip arms.

Another embodiment provides a flexible ankle foot orthotic method for treating foot drop steppage gait comprising the steps of providing a soft brace; affixing an ankle component of the soft brace to an ankle; affixing tension components to footwear; adjusting, at an attachment component, tension between the ankle component and the footwear; wherein the tension is adjustable before and during wearing. Included embodiments comprise threading each of the tension components through a serpentine path in the attachment components. In yet further embodiments, the ankle component comprises three ankle attachment components and three footwear attachment components. In related embodiments the footwear comprises more than three footwear attachment components and more than three footwear attachment components. For further embodiments the threading comprises feeding the tension component through at least one aperture, through multiple clip notches around at least one prong, and into at least one rear clip. In ensuing embodiments, adjusting tension comprises a single pulling motion with one hand on at least one tension components. Yet further embodiments comprise trimming excess of the tension components, wherein each of the attachment components is released by pressing a button of a clip of the attachment component with a single finger or thumb. For more embodiments, at least one attachment component is affixed to an outer side of the ankle component. Continued embodiments include at least one attachment component is a side attachment component affixed to one of a wedge, midsole, or heel portion of the footwear, whereby types of the footwear are increased. Additional embodiments comprise modifying the footwear with footwear attachment components of the soft brace.

A yet further embodiment provides a flexible ankle foot orthotic system for treating foot drop steppage gait comprising the steps of providing a soft flexible brace comprising an ankle component; a footwear component; a plurality of ankle attachment components disposed on the ankle component; a plurality of footwear attachment components disposed on the footwear component; and elastic tension components between the ankle attachment component and the footwear attachment component; modifying the footwear with the footwear attachment components of the soft brace; inserting a foot into the footwear component; affixing the ankle component to an ankle; affixing the elastic tension components; adjusting, at an ankle attachment component, tension between the ankle attachment component and the footwear component; and trimming excess of the tension components; wherein the tension is adjustable with one hand before and during wearing, and dorsiflexion and eversion support is provided for the foot, allowing a full range of motion of the foot and the ankle.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a perspective view of a first embodiment showing the (3) connection points on the left foot configured in accordance with the present invention.

FIG. 2 illustrates views of a first embodiment configured in accordance with the present invention.

FIG. 3 illustrates additional views of a first embodiment configured in accordance with the present invention.

FIG. 4 is a detail view of side clip components for the first embodiment depicting D-ring and fastener detail configured in accordance with the present invention.

FIG. 5 is a detail view of forward components for the first clip embodiment depicting D-ring and fastener detail configured in accordance with the present invention.

FIG. 6 is a perspective view of the first embodiment depicting D-ring and fastener detail configured in accordance with the present invention.

FIG. 7 is a depiction of scale cord connector clip components of a first embodiment configured in accordance with the present invention.

FIG. 8 depicts drawings of an ankle cuff embodiment configured in accordance with the present invention.

FIG. 9 is a perspective view of a second embodiment configured in accordance with the present invention.

FIG. 10 illustrates additional views of a second embodiment configured in accordance with the present invention.

FIG. 11 illustrates additional views of a second embodiment configured in accordance with the present invention.

FIG. 12 is a detail view of second embodiment clip attachment components configured in accordance with the present invention.

FIG. 13 is a detail view of a side clip second embodiment attachment component configured in accordance with the present invention.

FIG. 14 is a detail view of forward components of a second embodiment configured in accordance with one embodiment of the present invention.

FIG. 15 is a detail view of components configured in accordance with a second embodiment of the present invention.

FIG. 16 is a depiction of scale cord connector clip components configured in accordance with a third embodiment of the present invention.

FIG. 17 is a depiction of cord connector clip components configured in accordance with a fourth embodiment of the present invention.

FIG. 18 depicts drawings of an ankle component second embodiment configured in accordance with the present invention.

FIG. 19 is a depiction of foot motions in accordance with the present invention.

FIG. 20 is a flow chart of a method configured in accordance with embodiments of the present invention.

### DETAILED DESCRIPTION

Activity levels of users have changed over the past 30-40 years. With the increase in foot, knee, ankle, hip and back surgeries, as well as medication advances for diseases to slow its progress or symptoms, more people are ending up with foot drop who don't necessarily have a debilitating condition/disease that would force them to be less active or disabled. The prior art has focused on getting people who move very little to at least be able to complete small daily activities. This invention focuses on getting a person with foot drop to the same levels of activity they may have had prior to their condition occurring, such as working full time or participating in activities, such as exercising, running, hiking, walking on uneven terrain, gardening, athletics, or riding a bicycle. The failure of prior art has resulted in long felt but unresolved need that has been experienced by those with foot drop. The combination of pieces, parts, and functionality of this invention is unique providing increased reliability, confidence and the amount of activity that is possible.

Distinguishing features that set this invention apart from others include the three individual secure, adjustable elastic cord connections from the ankle cuff to the shoe, allowing for access to a wider variety of footwear without laces, eyeholes or Velcro® strips. The side clip increases the footwear options further by securing the heel allowing access to certain types of footwear that would not be possible to wear otherwise, where the heel of the foot would fall out of the shoe due to foot paralysis and lack of muscle control, including dress shoes and slippers. The three separate attachments also allow the user to adjust the tension to accommodate for their specific severity of eversion (an outward foot tilt), which is another tripping hazard, as well as for comfort and support for longer term wear, increasing confidence and the amount of activity that is possible.

In embodiments, other considerations include attaching the side attachment component to the wedge, midsole or heel portion of the footwear sole to further increase the types of shoes that can be worn; the location of the side attachment component on the ankle component and the footwear could vary slightly based on users condition and comfort; the ankle component or the footwear attachments can fasten in various ways using metal hardware, plastic hardware, Velcro® or Velcro® strap; additional connection points can be mounted on the ankle component and the footwear for added ankle support. Other embodiments include attaching directly to the shoe without the need for an ankle cuff if the footwear, such as a boot, has a sufficient high ankle component extending above the ankle.

Embodiments of the invention provide the ability to walk and be physically active in many more of life's activities without the concern of tripping. They address foot tilting to the side, which is a tripping hazard for those with foot drop. They provide secure connection to the shoe. Versus rigid braces, embodiments allow user flexibility of the ankle and provide the opportunity to strengthen/use muscles regularly by walking as naturally as possible. They are comfortable to wear for extended periods of time (9-10 hours plus per day). They work with a larger selection of shoes with modification. While for embodiments it is recommended to have a shoe modification professional, such as a cobbler, make appropriate modifications, these can be done by the wearer. They are suitable for distribution directly to the wearer; however distribution through industry professionals such as an orthotist, podiatrist, or pedorthist, would allow the wearer to receive advice and support on the product.

Embodiments comprise multiple attachment components located high on the brace to provide sufficient support; only one connector clip may not provide sufficient support. For embodiments, attachment components can be clips or buckles. ‘Real-time’ adjustable tension enables quick, convenient alterations during wearing to ensure maximum tension and comfort. Known products utilize the laces, the eyelets with hooks or Velcro® to connect the shoe to the soft brace, none of which provides a secure connection and supports foot drop for long periods of time. Laces tighten, allowing the connection to loosen, hooks can come out of the eyelets easily, and Velcro® does not hold tightly.

The side elastics of embodiments address the foot tilt and assists in keeping the heel of the foot comfortably in the shoe without concern of the heel coming out of the shoe during activity. The quantity of attachments, type, and location adds support and stability. Tension remains strong throughout the day and can be adjusted over longer periods of time as needed. Elastics can be easily replaced when needed. All connection points are secure. Embodiments provide the ability to return to a normal lifestyle, work full time, or participate in athletic activities that currently may not be easy or possible to participate in. The user has the ability to wear normal size shoes as well as a variety of shoe types, such as dress shoes, heels, boots, sandals, sneakers, slippers, etc with some modification by the wearer, or industry professional.

### GLOSSARY

Orthotist, a healthcare professional providing and/or supervising the assessment, treatment plan development, and implementation, follow-up, and practice management of people using prescribed orthotics.

Pedorthist a professional with specialized training to modify footwear and employ supportive devices to address conditions which affect the feet and lower limbs.

Cobbler is a professional who specializes in making, mending and modifying footwear.

Orthosis refers to an externally applied device used to modify structural and functional characteristics of the limbs or spine to correct alignment or provide support (pl. orthoses).

Orthotics is the medical field concerned with the design, manufacture, and application of orthoses.

Cord connector clip and/or clip attachment components provide attachment connections between one or more elastic components and an ankle or foot brace component.

Dorsum refers to the back of the appendage.

Eversion is the movement where the sole of the foot tilts away from the median plane of the body, or outward.

Inversion is the movement where the sole of the foot tilts towards the median plane of the body, or inward.

Dorsiflexion is movement where the toes are brought upward, closer to the shin, decreasing the angle between the dorsum of the foot and the leg.

Plantarflexion is opposite dorsiflexion, or movement downward, extending the toes, increasing the angle between the sole of the foot and the leg.

Pronation is a normal part of the gait cycle which helps provide shock absorption at the foot, where the outside part of the heel makes initial contact with the ground and the foot “rolls” inward to come in complete contact with the ground and can support the body's weight without any problem distributing the forces of impact.

Overpronation of the foot refers to “rolling” of the foot slightly inward where the arch of the foot flattens; the body weight is distributed unevenly and inefficiently, causing excessive stress and pressure on the soft tissues of the foot.

Supination or underpronation of the foot refers to “rolling” of the foot slightly outwards, where the high arches of the foot causes excessive stress and pressure of impact to be concentrated on the outside portion of the foot.

Steppage gait is a style of walking where the leg is raised abnormally high due to a disorder.

Quarter area of footwear is the rear portion of a shoe comprising the part that covers the heel and is often joined at the back by a seam.

Vamp area of footwear is the part of a shoe upper covering especially the forepart of the foot and sometimes also extending forward over the toe or backward to the seam.

As mentioned, objects of the invention are to overcome the shortfalls of current devices. The present invention is an ankle foot orthosis device that provides a user with the ability to walk with a natural gait while expanding the footwear options for activities beyond what is currently available. The invention consists of a soft flexible fabric ankle cuff that wraps around the ankle and is secured with, in embodiments, a Velcro® strap. The ankle cuff is made of a non-stretch breathable material that is comfortable and provides support for long term use. The ankle cuff can be worn over a sock or directly against the skin. Securely attached to the ankle cuff are three cord connector clip tops located to align with the three shoe attachments. The footwear options that can be worn with the invention include an increased variety of normal sized shoes, including, but not limited to, heels, sneakers, slippers, sandals, or boots that may or may not have laces or eyeholes allowing the user many more options. Footwear can be modified prior to use by a shoe manufacturer or distributor, by a shoe modification professional or by the user, where a D-ring is securely attached to the shoe. The locations of the footwear attachments are designed for optimum support of foot drop and can accommodate various shoe designs. There are two attachments on the front of the footwear, located on the forepart of the vamp. In embodiments, the third side attachment is located on a rear portion of the quarter area of outer side of the footwear. Each attachment comprises a D-ring, elastic cord, and a cord connector clip with a top and bottom section. A ⅛″ elastic cord is threaded on one side of the cord connector clip bottom through the D-ring on the footwear to the second side of the cord connector clip bottom utilizing the apertures, clip notches, prongs, and rear clip-ins. These custom cord connector clips allow for the elastic cord to be easily adjusted by the user (by a single finger and thumb) at any time by pulling the elastic out of the rear clip ins and clip notches and pulling/releasing the cord to the preferred length, and then rethreading. The cord connector clip bottom is held with the user's fingers and pulled upward to snap securely into the cord connector clip top. Once attached, the invention is ready for use and can be detached at any time by pushing the button with the thumb or finger for a quick release. The cord connector clip and the elastic cord remain attached to the shoe and can be quickly and easily reattached to the cord connector clip at any time without any additional preparation. The cord connector clips have been designed to be small, discrete, and easy to use. The elastic cords and the footwear connection points allow sufficient tensile strength to support the foot without the possibility of breakage during wear, while allowing sufficient movement of the foot for natural walking and running activities. All components may be selected from a variety of colors to coordinate with the shoes/socks that one is wearing to increase the aesthetic appearance. This invention provides a solution that is unique, incorporating all of the following features in one invention: soft, lightweight, discrete, easy to use, comfortable, inexpensive, providing dorsiflexion and eversion support, is durable and suitable for long-term wear, can be used with a large variety of shoe types and styles, while allowing for full range of motion of the foot and ankle, providing increased flexibility and mobility, increasing the types of activity possible for those with foot drop.

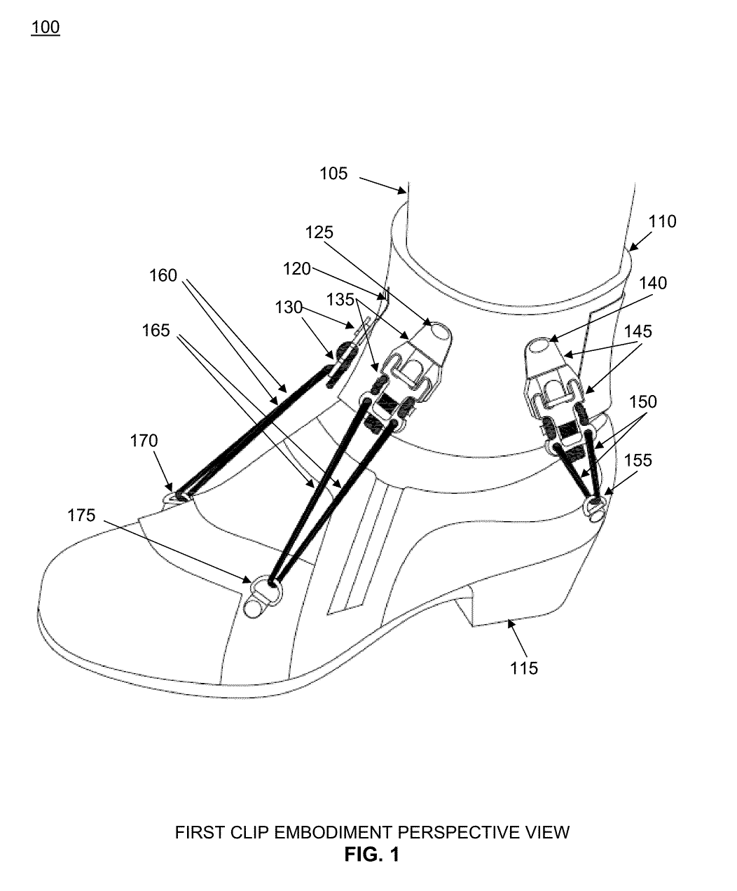

FIG. 1 shows a perspective view 100 of a first embodiment depicting leg 105; comprising ankle component 110; shoe 115; first front ankle attachment 120; second front ankle attachment 125; first front ankle clip component 130; second front ankle clip component 135; side attachment 140; side clip component 145; side elastic components 150; shoe side attachment located on the quarter area of the footwear 155; first front elastic components 160; second front elastic components 165; first front shoe attachments 170; and second front shoe attachments located on the vamp area of the footwear 175.

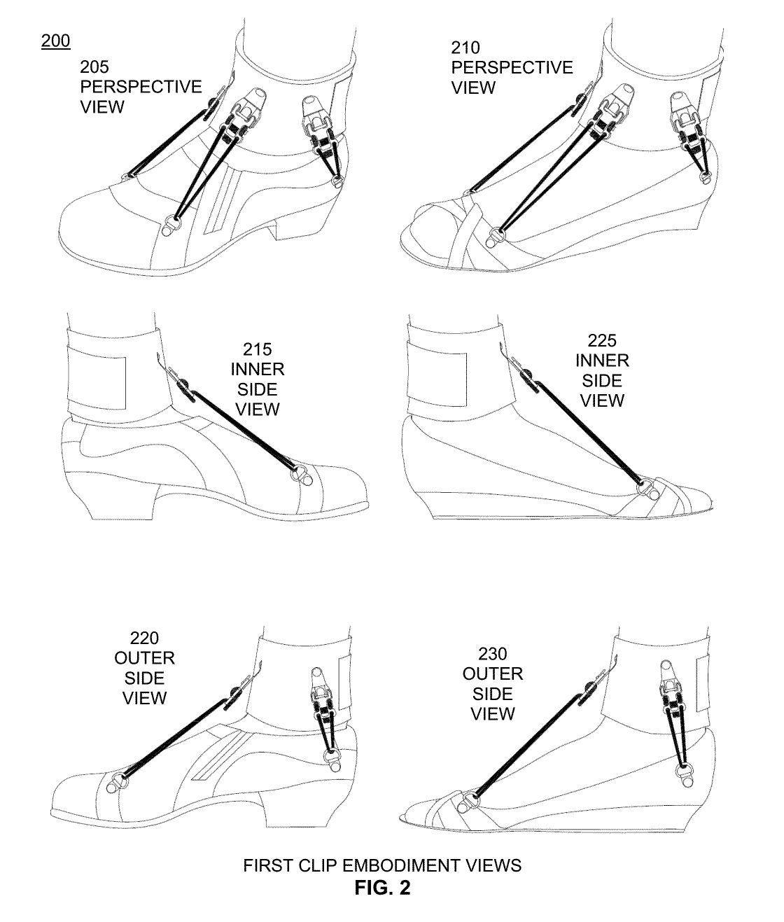

FIG. 2 illustrates views 200 of a first embodiment. Views are first perspective view 205; second perspective view 210; inner side view 215 of first perspective view 205; outer side view 220 of first perspective view 205; inner side view 225 of second perspective view 210; and outer side view 230 of second perspective view 210.

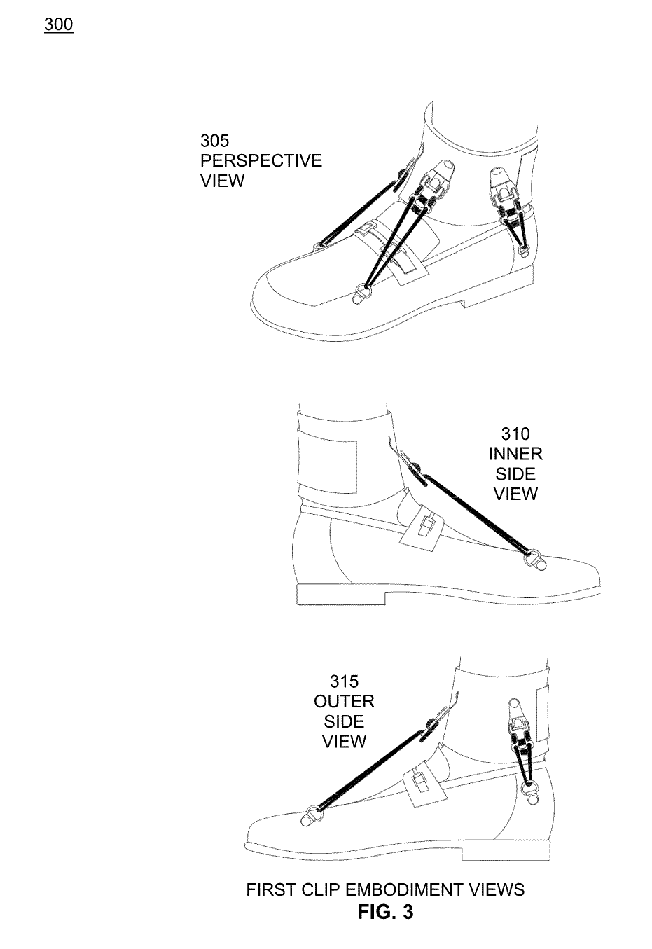

FIG. 3 illustrates additional views 300 of a first embodiment. Views are third perspective view 305; inner side view 310; and outer side view 315.

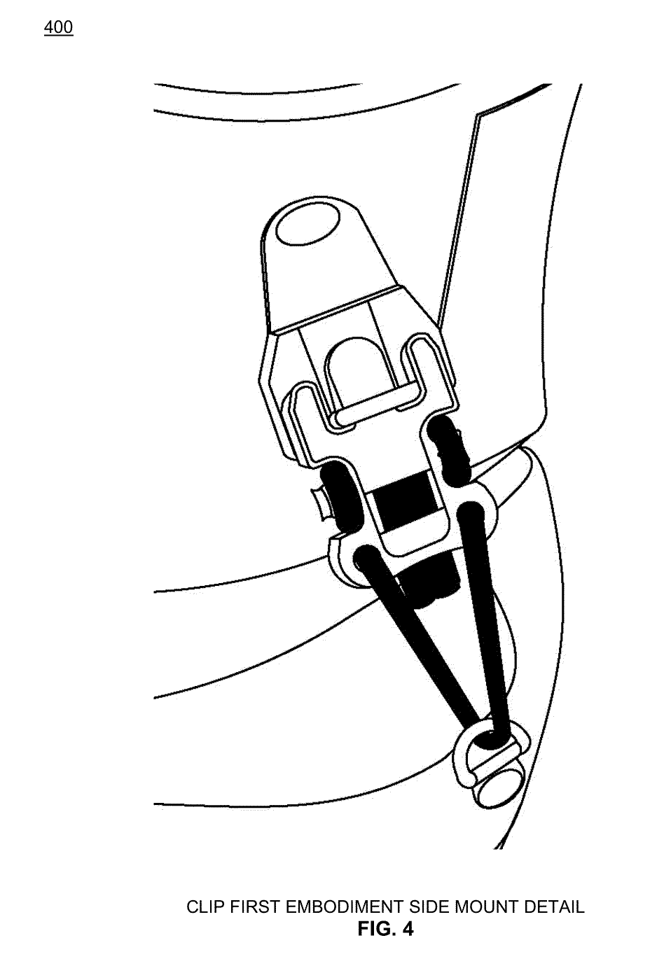

FIG. 4 is a detail view 400 of a side clip components for the first embodiment depicting D-ring and fastener detail.

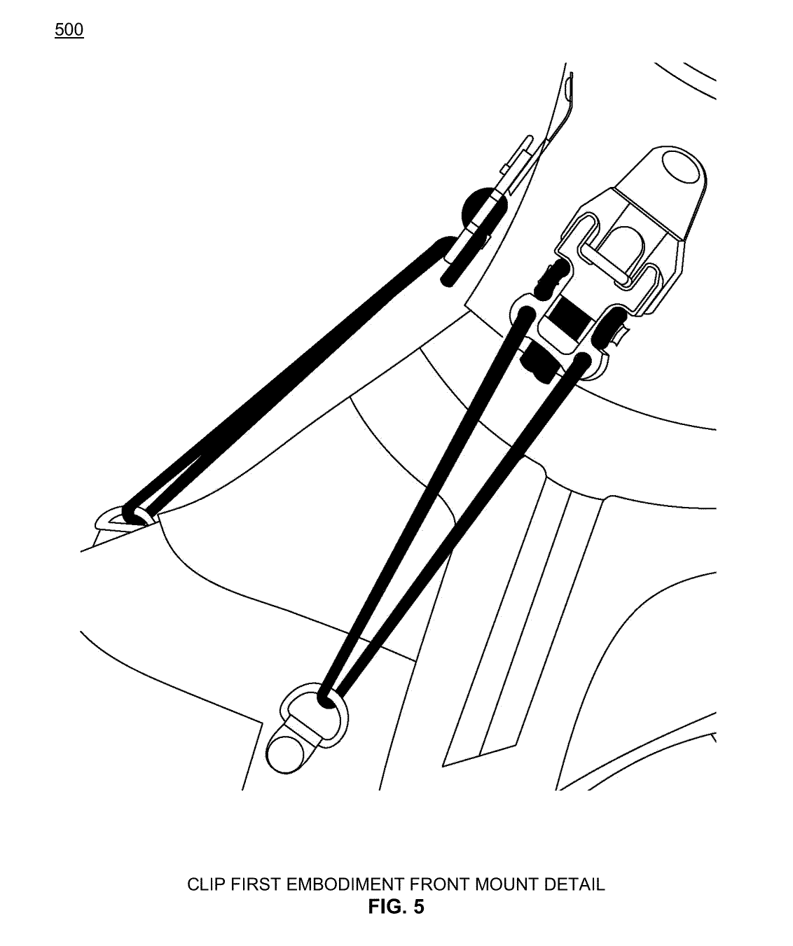

FIG. 5 is a detail view 500 of forward components for the first clip embodiment depicting D-ring and fastener detail.

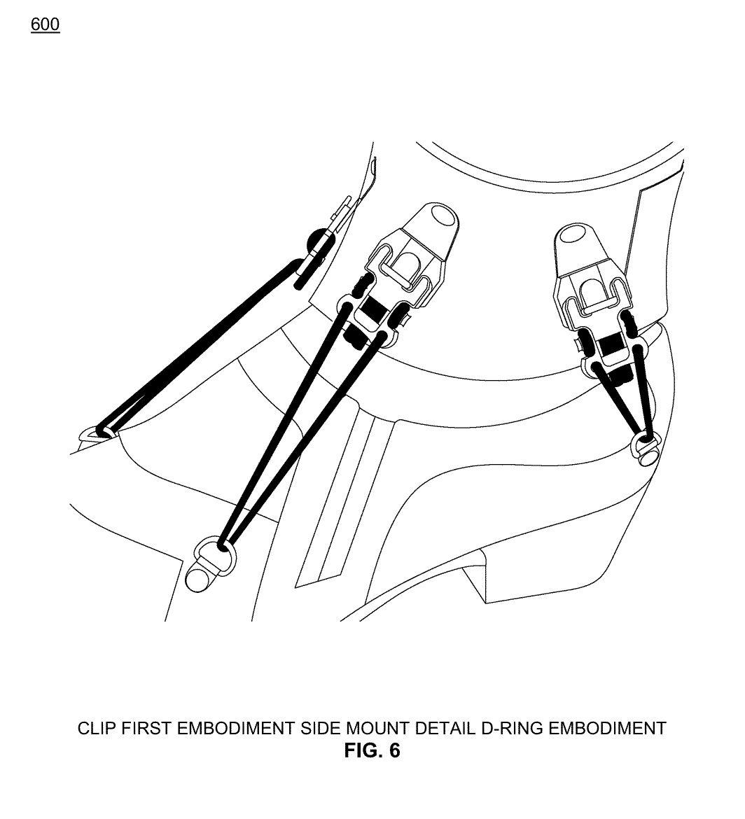

FIG. 6 is a perspective view 600 of the first embodiment depicting D-ring and fastener detail.

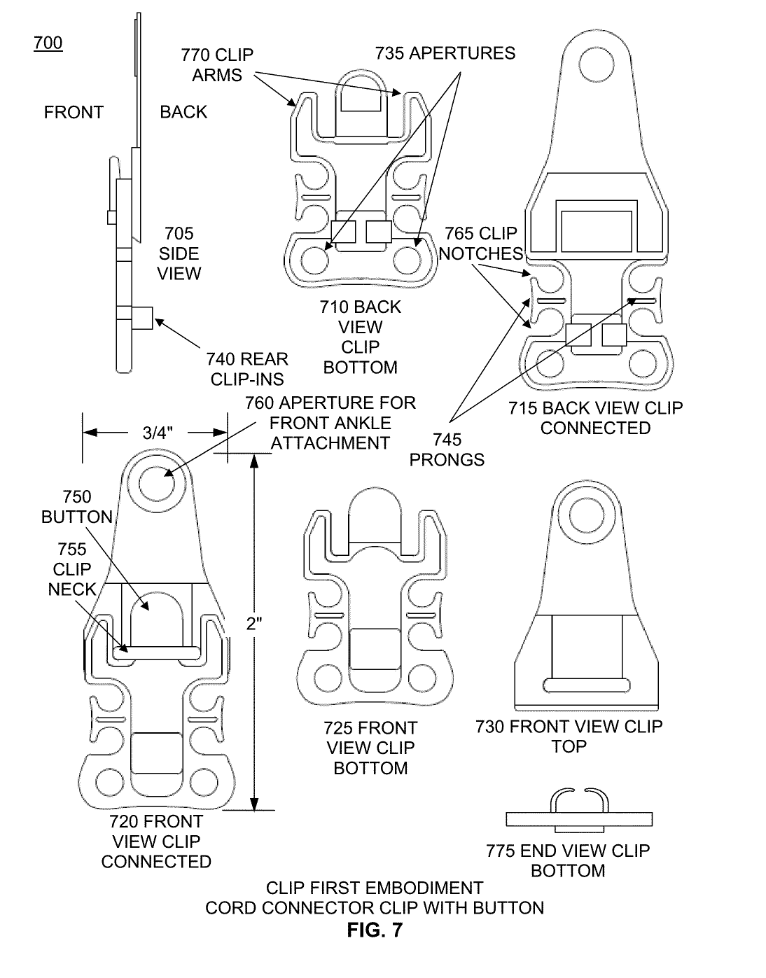

FIG. 7 is a detail scale view 700 of cord connector clip components for a first embodiment. Views include first embodiment side view 705, first embodiment back view clip bottom 710; first embodiment back view clip connected 715; first embodiment front view clip connected 720 reflecting the overall dimensions of 2″ long and ¾″ wide; first embodiment front view clip bottom 725; and first embodiment front view clip top 730. Each clip includes two apertures 735, two rear clips-ins 740 for holding extra elastic cord length and for adjusting tension, and prongs 745, button for clip connection and thumb press release for clip disconnection 750; clip neck to hold clip securely fastened 755; aperture for front ankle attachment 760; clip notches to hold, for example, ⅛ inch diameter elastics securely 765; clip arms 770; and end view of clip bottom 775.

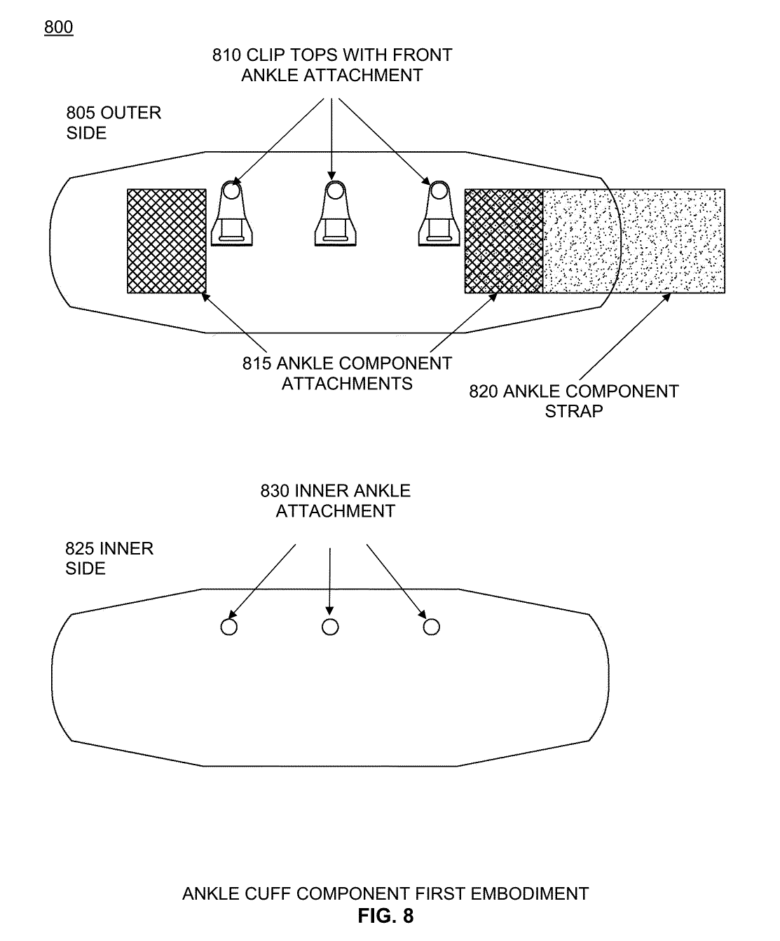

FIG. 8 depicts drawings 800 of an ankle component first embodiment comprising outer side 805. Shown on outer side 805 are clip tops with front ankle attachments 810; ankle component attachments 815; and ankle component strap 820. Inner side 825 has inner ankle attachments 830 shown.

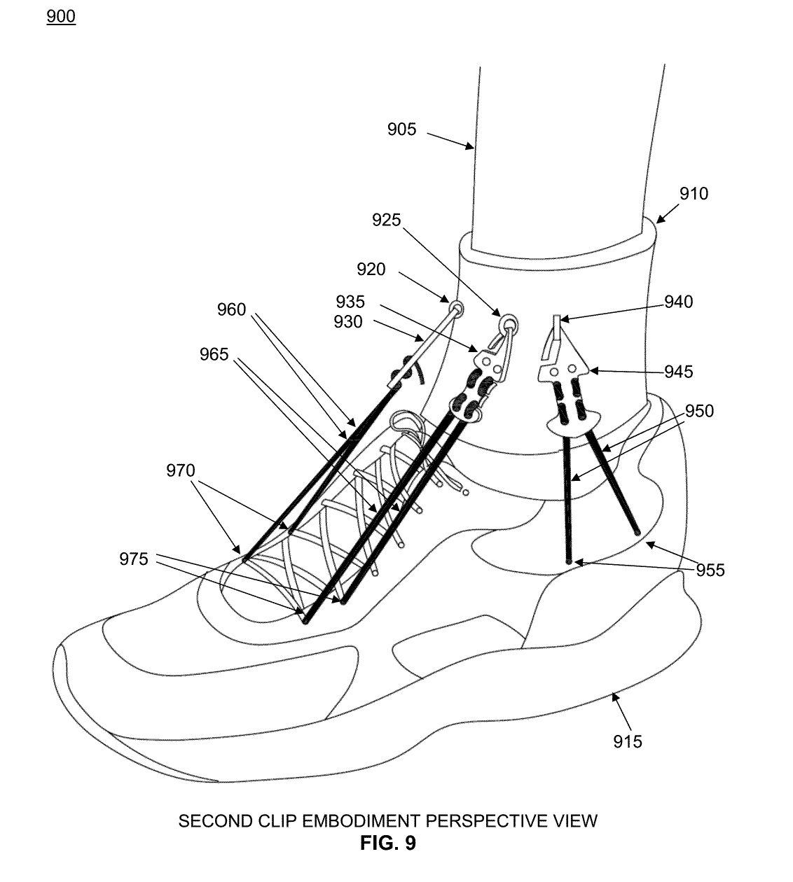

FIG. 9 shows a perspective view 900 of a second embodiment utilizing an o-ring and hook attachment depicting leg 905; comprising ankle component 910; shoe 915; first front ankle attachment 920; second front ankle attachment 925; first front ankle clip component 930; second front ankle clip component 935; side attachment 940; side clip component 945; side elastic components 950; first shoe side attachment showing an embodiment where holes are punched directly into the shoe disregarding the need for a D-ring 955; first front elastic component 960; second front elastic component 965; first front shoe attachments showing an embodiment utilizing the existing eyeholes in the sneakers 970; and second front shoe attachments utilizing the existing eyeholes in the sneaker 975.

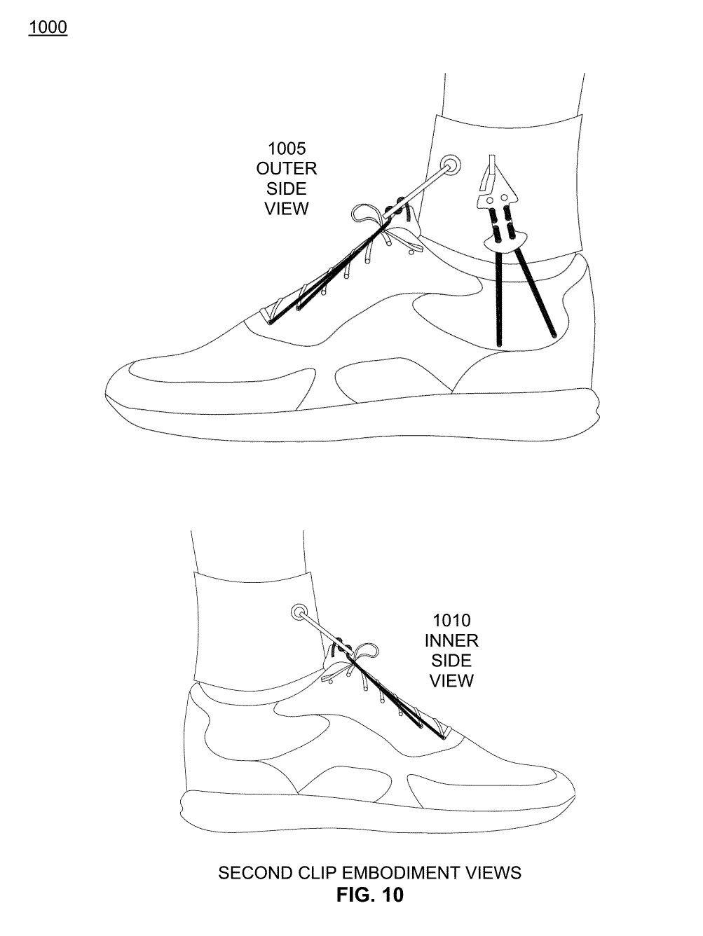

FIG. 10 illustrates views 1000 of a second embodiment. Views are outer side view 1005; and inner side view 1010.

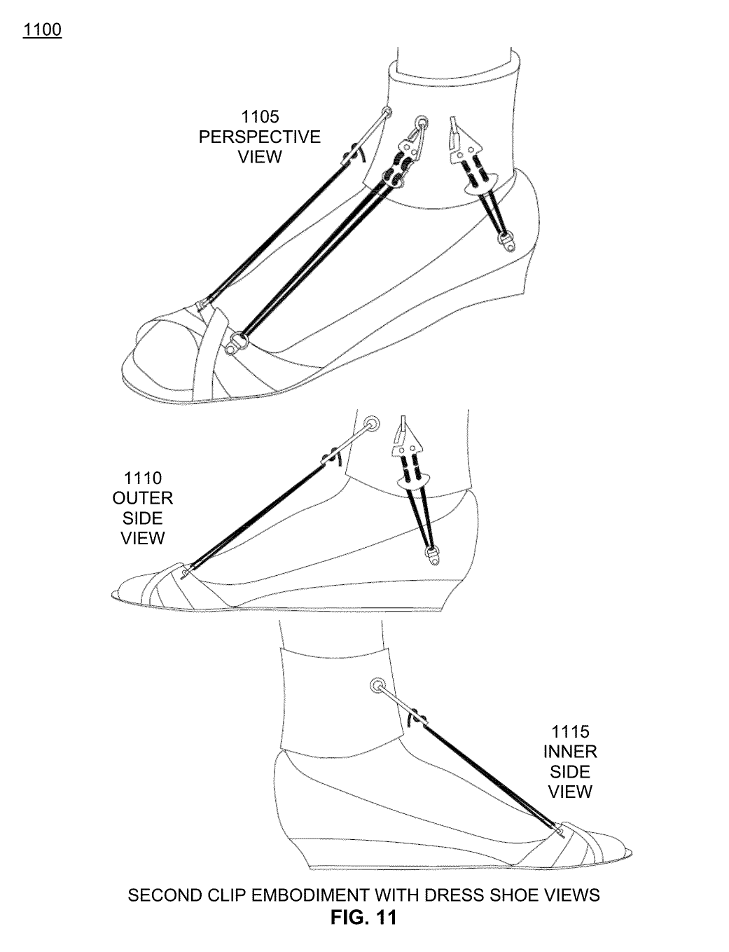

FIG. 11 illustrates views 1100 of a second embodiment. Views are perspective view 1105; outer side view 1110; and inner side view 1115.

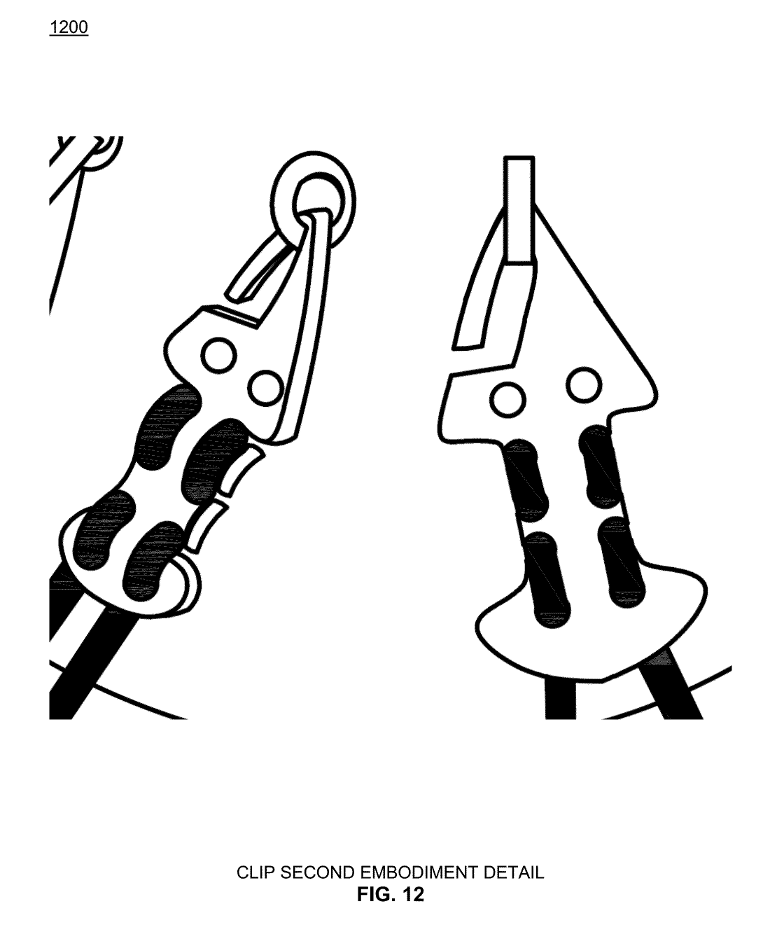

FIG. 12 illustrates a detail view of clip components 1200 for a second embodiment.

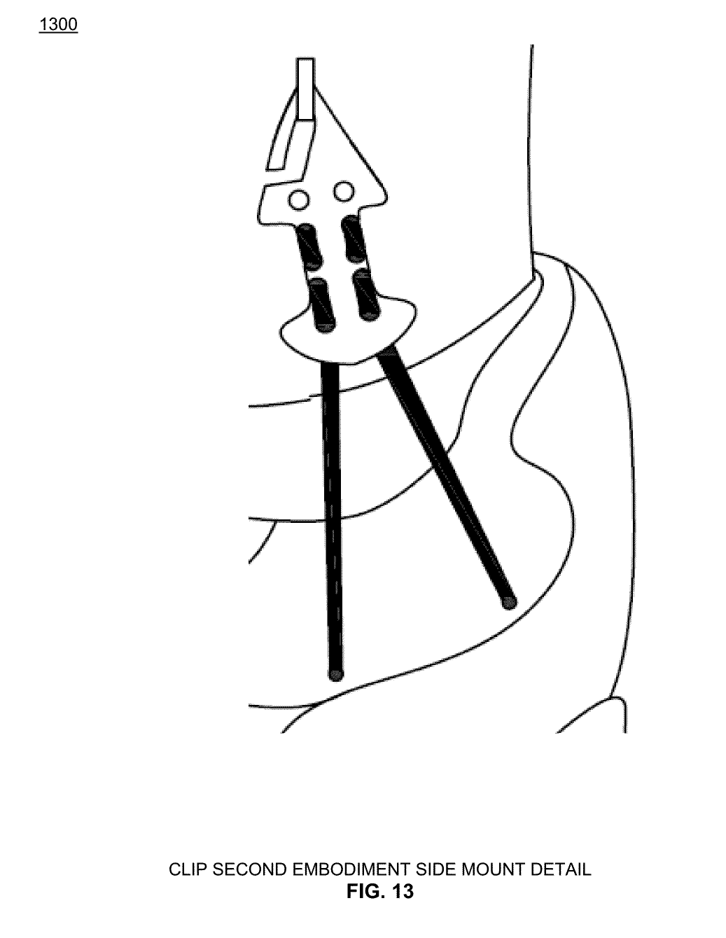

FIG. 13 is a detail view of an outer side clip component 1300 for a second embodiment.

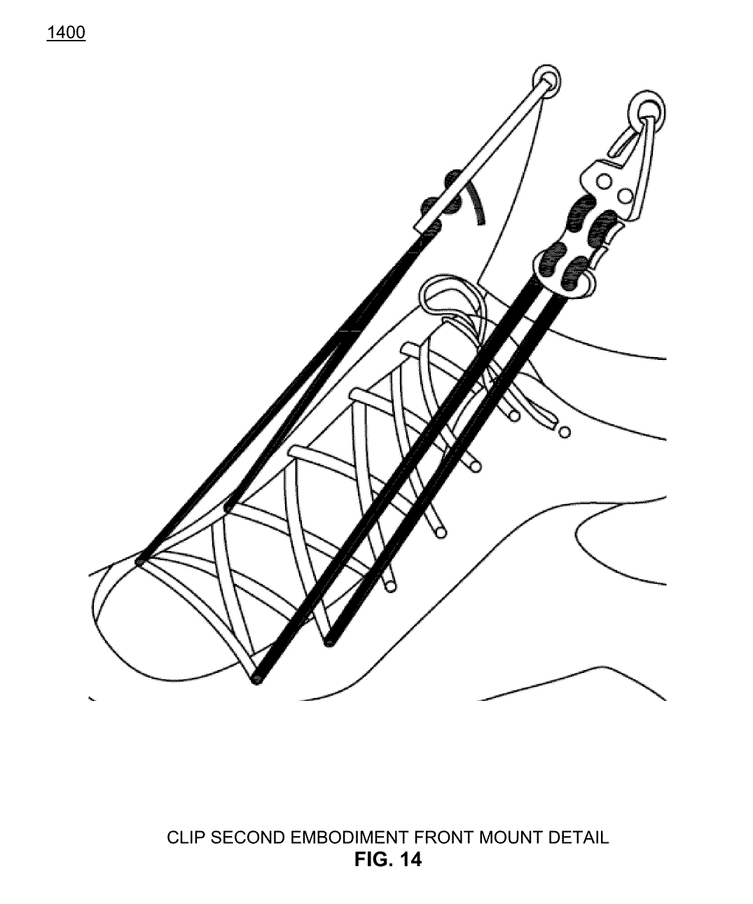

FIG. 14 is a detail view 1400 of forward components for a second clip embodiment.

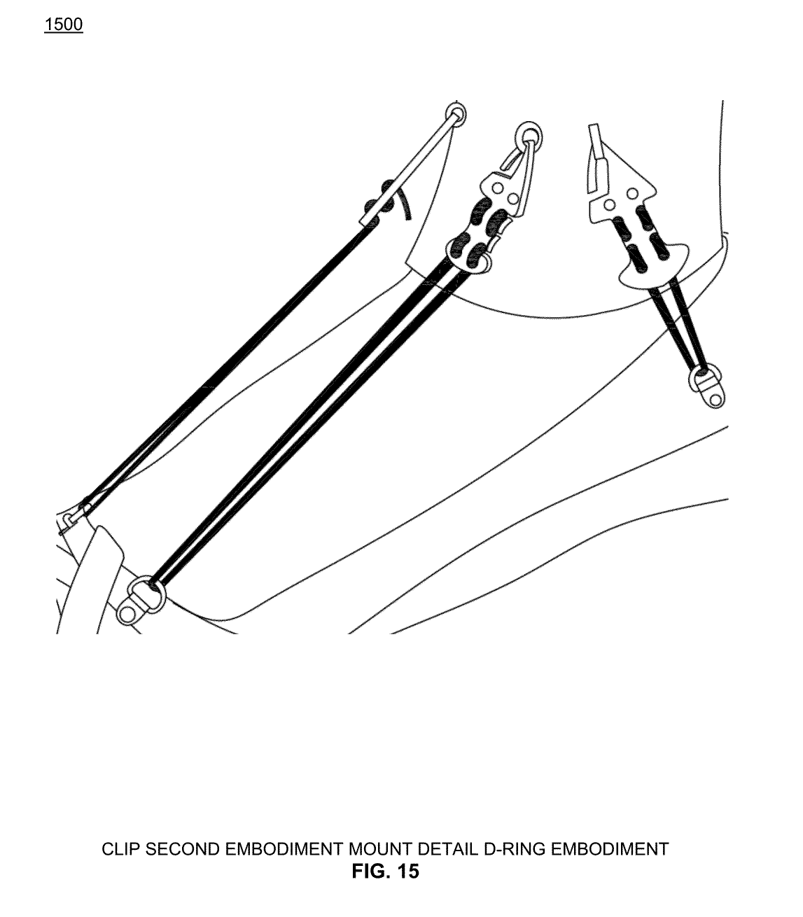

FIG. 15 is a perspective view 1500 of a third embodiment depicting D-ring detail.

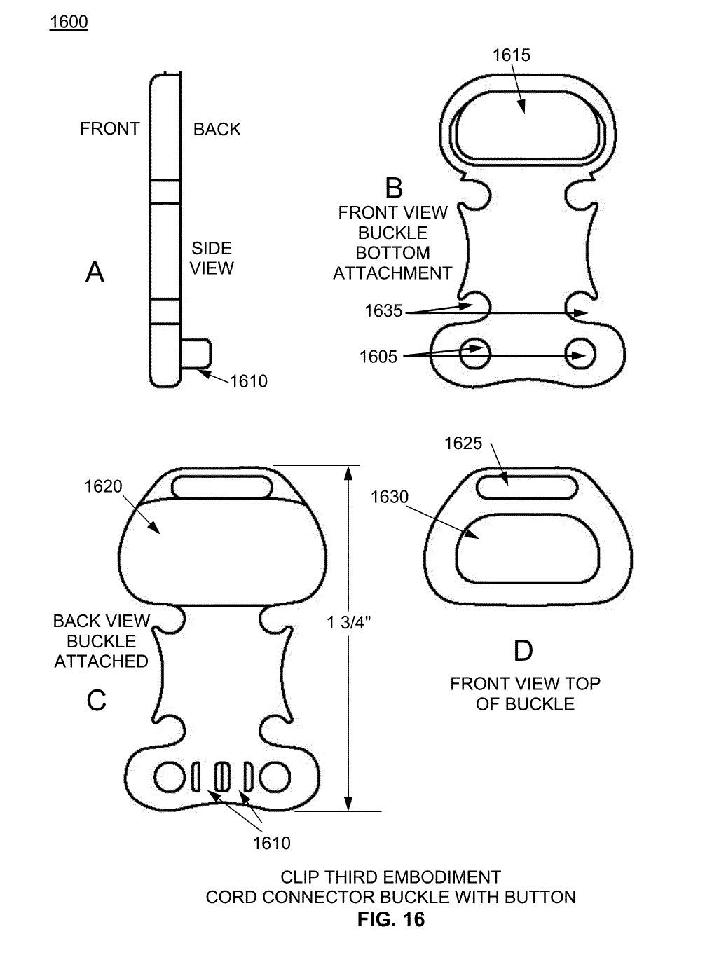

FIG. 16 is a detail scale view 1600 of cord connector clip components for embodiments. Four views include first embodiment side view A, first embodiment front view B, first embodiment back view C (with buckle D attached), and cord connector buckle button attachment D. Each clip includes two apertures 1605. Rear clips 1610 (for extra elastic length for adjusting) are on the back side of view B. They are shown in this view for placement information. Rear clips 1610 hold excess elastic. Push button 1615 is shown in view B. Back view C depicts button attachment D affixed 1620. Opening 1625 for attachment to brace and opening 1630 for button are shown in view D. Interior and exterior edges are rounded. For embodiments, the button is sized for a thumb to press. In embodiments, clip notches 1635 are large enough to hold two ⅛ inch diameter elastics securely.

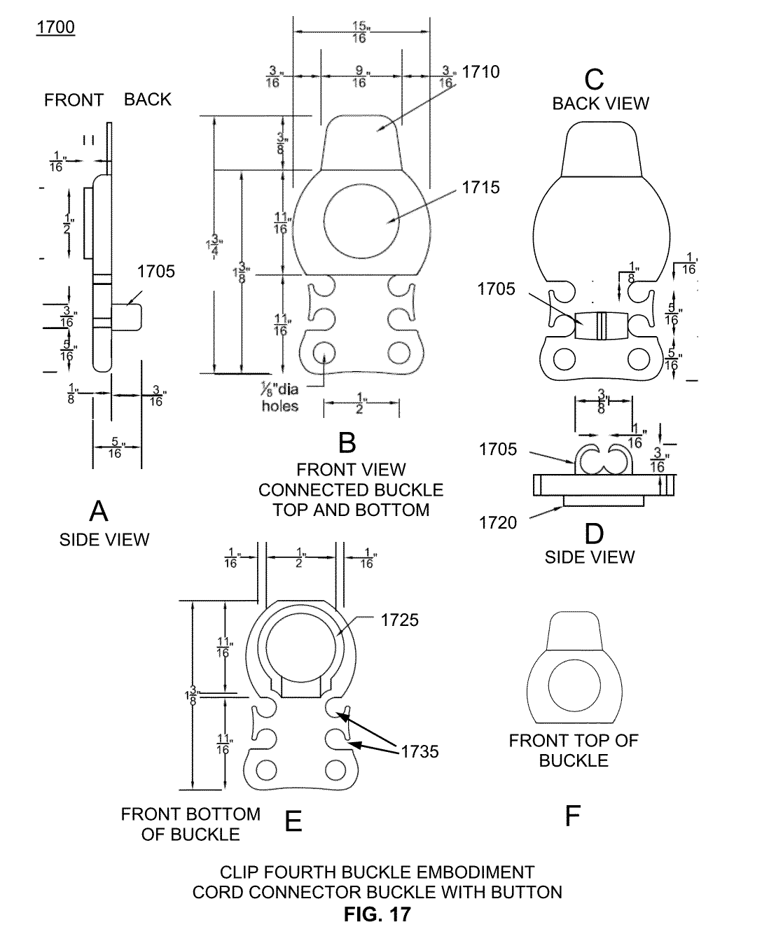

FIG. 17 is a detail view 1700 of another embodiment for cord connector buckle components. Six views include second embodiment side view A, second embodiment front view B, second embodiment back view C, bottom side view D, front bottom view E, and front top of cord connector buckle F. Rear clips 1705 for extra elastic length for adjusting are shown on side view A, back view C, and bottom side view D. Rear clips 1705 hold excess elastic. 1710 is the attachment to the ankle brace which can either be sewed or attached in some other fashion. Button, sized for a thumb, 1715 is shown in front view B. Side view D also depicts front-button 1720. Front bottom view E shows open space 1725 for a button. Interior and exterior edges are rounded. For embodiments, clip notches 1735 are large enough to hold two ⅛ inch diameter elastics securely.

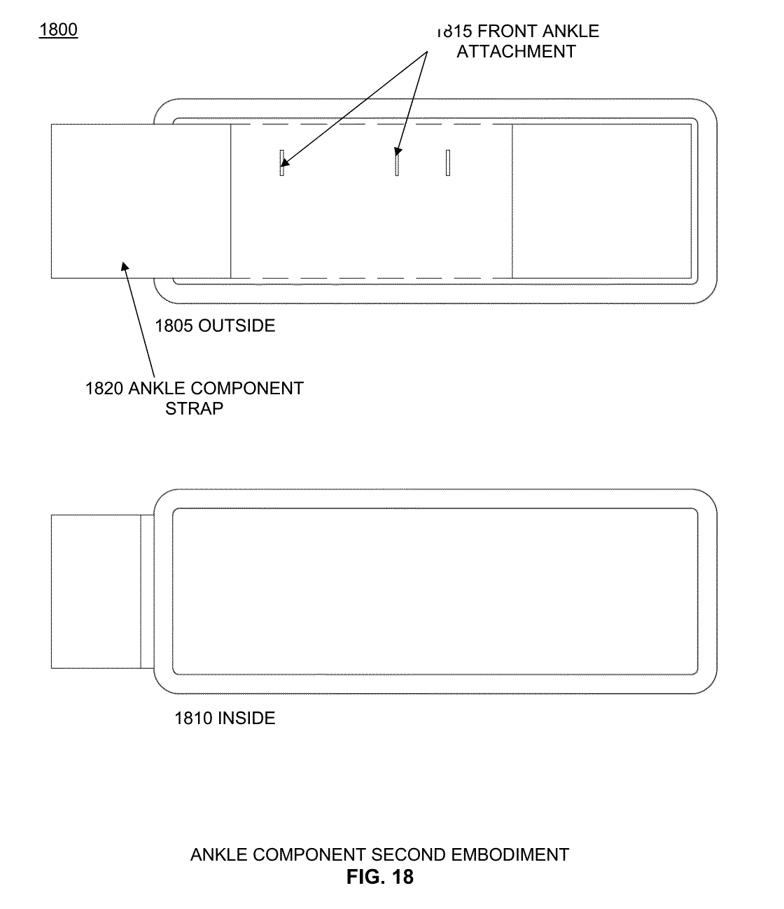

FIG. 18 depicts drawings 1800 of an ankle component second embodiment comprising outer side 1805 and inner side 1810. First front ankle attachment is shown 1815, and ankle component strap 1820.

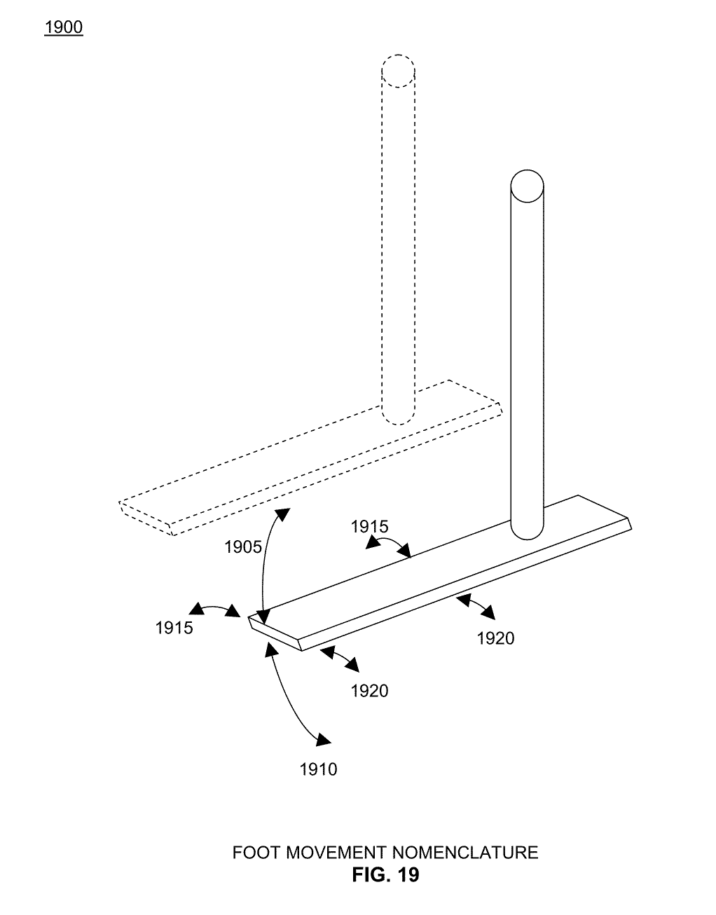

FIG. 19 depicts movement of the foot 1900 including dorsiflexion 1905; plantarflexion 1910; inversion 1915; and eversion 1920. The stages of walking consist of repeated gait cycles. The gait cycle contains two main phases, stance phase and swing phase. The two front clip connections lift the front of the foot up providing dorsiflexion during the swing phase. On the end of the swing phase when entering the stance phase, surprisingly good plantarflexion is possible due to the tensile strength of the elastic cords and the secure connection. With foot drop, ankle weakness may be apparent where ankle eversion causes a trip hazard over the side of the shoe. The side connection allows the side/back of the foot to be lifted slightly to avoid this hazard surprisingly well. The side clip connection also allows for an increased footwear selection and increased activity by keeping the heel in the footwear comfortably.

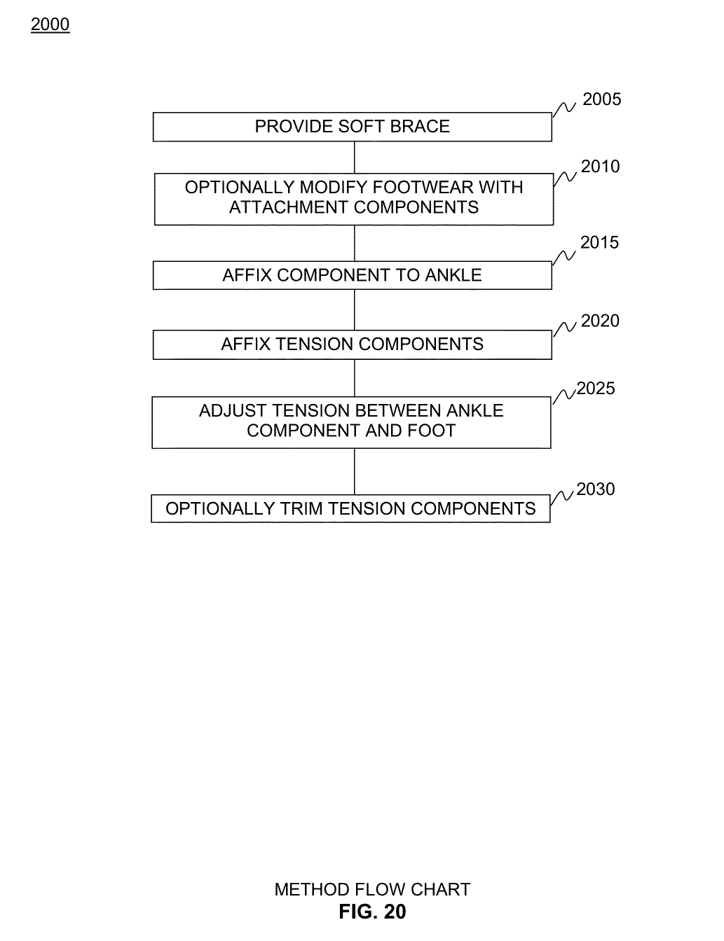

FIG. 20 is a flow chart of a method 2000 comprising the steps of providing a soft brace 2005; optionally modifying footwear with attachment components 2010; affixing a component to an ankle 2015; affixing tension components 2020; adjusting tension between ankle component and foot 2025; and optionally trimming tension component excess 2030.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A flexible ankle foot orthosis device for treating foot drop steppage gait and providing ankle support comprising:
an ankle component;
a foot component;
a plurality of ankle attachment components disposed on said ankle component;
a plurality of foot attachment components disposed on said foot component; and
elastic tension components between said ankle attachment components and said foot attachment components;
wherein said elastic tension components at said ankle attachment components are adjustable before and during wearing.

2. The device of claim 1 comprising three said ankle attachment components disposed on said ankle component and three said foot attachment components disposed on said foot component.

3. The device of claim 2 wherein each of said ankle attachment components comprises a clip configured to receive at least one said elastic tension component.

4. The device of claim 3 wherein each clip has a serpentine path retaining said at least one elastic tension component.

5. The device of claim 4 wherein said serpentine path comprises at least one aperture, at least one prong, and at least one rear clip, whereby said at least one elastic component can be drawn to adjust and maintain tension between said ankle component and said foot component.

6. The device of claim 4 wherein said serpentine path comprises two apertures, two rear clips, and two prongs.

7. The device of claim 1 wherein said adjustment is accomplished by a single pulling motion with one hand on at least one said elastic tension component.

8. The device of claim 1 wherein said ankle attachment component comprises a clip top component and a clip bottom component.

9. The device of claim 8 wherein said clip top component is affixed to said ankle component, said clip bottom component removably attaches to said clip top component and comprises two clip arms.

10. A flexible ankle foot orthotic method for treating foot drop steppage gait comprising the steps of:
providing a soft brace;
affixing an ankle component of said soft brace to an ankle;
affixing tension components to footwear;
adjusting, at an attachment component, tension between said ankle component and said footwear;
wherein said tension is adjustable before and during wearing.

11. The method of claim 10 comprising threading each of said tension components through a serpentine path in said attachment components.

12. The method of claim 10 wherein said ankle component comprises three ankle attachment components and three footwear attachment components.

13. The method of claim 10 wherein said footwear comprises more than three footwear attachment components and more than three footwear attachment components.

14. The method of claim 10 wherein said threading comprises feeding said tension component through at least one aperture, through multiple clip notches around at least one prong, and into at least one rear clip.

15. The method of claim 10 wherein said adjusting tension comprises a single pulling motion with one hand on at least one said tension components.

16. The method of claim 10 comprising trimming excess of said tension components; and
wherein each of said attachment components is released by pressing a button of a clip of said attachment component with a single finger or thumb.

17. The method of claim 10 wherein at least one said attachment component is affixed to an outer side of said ankle component.

18. The method of claim 10 wherein at least one said attachment component is a side attachment component affixed to one of a wedge, midsole, or heel portion of said footwear, whereby types of said footwear are increased.

19. The method of claim 10 comprising modifying said footwear with footwear attachment components of said soft brace.

20. A flexible ankle foot orthotic system for treating foot drop steppage gait comprising the steps of:
providing a soft flexible brace comprising an ankle component; a footwear component; a plurality of ankle attachment components disposed on said ankle component; a plurality of footwear attachment components disposed on said footwear component; and elastic tension components between said ankle attachment component and said footwear attachment component;
modifying said footwear with said footwear attachment components of said soft brace;
inserting a foot into said footwear component;
affixing said ankle component to an ankle;
affixing said elastic tension components;
adjusting, at an ankle attachment component, tension between said ankle attachment component and said footwear component; and
trimming excess of said tension components;
wherein said tension is adjustable with one hand before and during wearing, and dorsiflexion and eversion support is provided for said foot, allowing a full range of motion of said foot and said ankle.
